(19) United States
(12) Patent Application Publication
(10) Pub. No.: US 2019/0350969 A1
(43) Pub. Date: Nov. 21, 2019

(54) METHODS AND COMPOSITION FOR TREATING NITRIC OXIDE DEFICIENCIES

(71) Applicant: Shawn J. Green, Los Angeles, CA (US)

(72) Inventor: Shawn J. Green, Los Angeles, CA (US)

(21) Appl. No.: 16/416,734

(22) Filed: May 20, 2019

Related U.S. Application Data
(60) Provisional application No. 62/674,008, filed on May 20, 2018.

(51) Int. Cl. A61K 33/00; A61K 9/08; A61P 9/12

(52) U.S. Cl. CPC A61K 33/00; A61P 9/12; A61K 9/08

(57) ABSTRACT

Compositions, methods of manufacture, methods of improving dietary health, kits, devices, and uses for plasma activated water are provided. Plasma activated water may be used for improving nitric oxide levels, improving health and/or reducing symptoms of cardiometabolic syndrome in a subject. Compositions and methods for improving nitric oxide levels in a subject disclosed herein comprise consumption of a composition comprising dietary nitrate contained in plasma activated water. The health benefits of consuming plasma activated water include lowering blood pressure, lowering intraocular pressure, decreasing LDL/TG levels, restoring nitric oxide mediated cardiovascular benefits, including but not limited, restoring endothelium function and improving flow mediated dilation in nitric oxide deficient subjects. In addition, consumption of plasma activated water also improves oral health and reduces and/or prevents cancer burden. In an embodiment, plasma activated water may also benefit general health, as nitric oxide deficiencies correlate to numerous physiological aberrations, such as cancer.

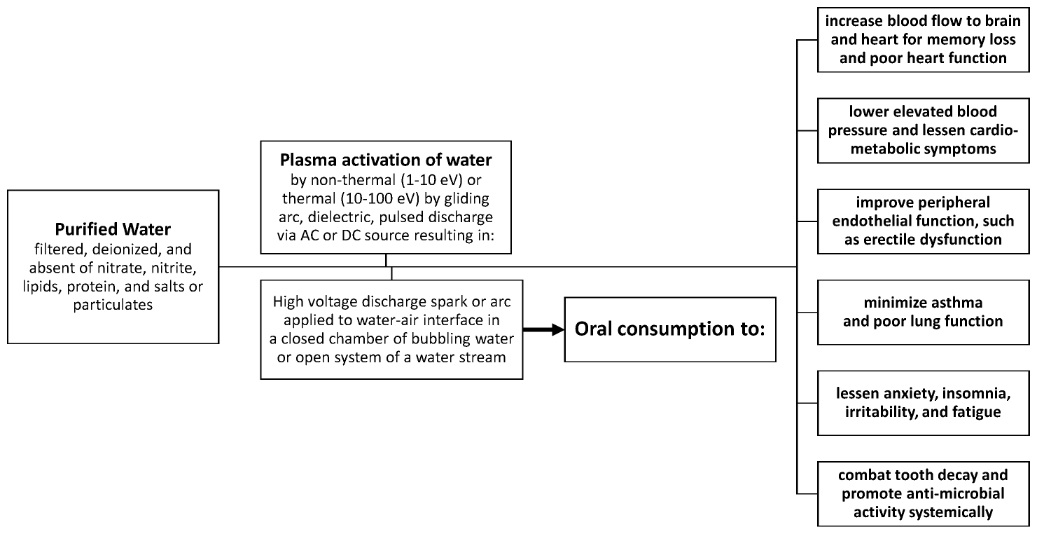

### CROSS-REFERENCE TO RELATED PATENT APPLICATIONS

This application claims priority to U.S. Provisional Patent Application Ser. No. 62/674,008, filed on May 20, 2018 the contents of which are incorporated by reference in their entireties for all purposes.

### TECHNICAL FIELD

Embodiments of the disclosure relate generally to methods and compositions for treating, preventing and/or managing symptoms of cardiometabolic syndrome and/or nitric oxide deficiencies. More particularly, the present invention relates to methods for treating and preventing unwanted symptoms of nitric oxide deficiency. The embodiments herein enable the improvement of health conditions, in particular, to alleviating cardiovascular health complications and enhance cardiovascular performance. Embodiments of the disclosed subject matter are relevant to the field of cardio-metabolic syndrome treatment and prevention using dietary plasma activated water.

### BACKGROUND OF THE INVENTION

### Plasma Activated Water

Methods for generating plasma activated water typically comprise the use of high voltage electricity. When applying high voltage as a dielectric barrier discharge, a corona or pulsed corona discharge, arc, spark, or gliding arc in the immediate proximity of the air-water interface reactive nitrogen and oxygen species are dissolved within the water. In brief, the nitrogen (N2) in air is cleaved, into two short-lived reactive single nitrogen molecules which react with reactive oxygen singlet species resulting in a mix of nitrites (NO2), nitrates (NO3). This process also creates unstable hydrogen peroxide (H2O2) and other nitrogen oxide intermediates (NOx), such as the short-lived nitric oxide (NO), which is immediately oxidized to form intermediates included the more stable nitrogen oxides, such as NO3 and NO2 resultants in the water. Accordingly, the mechanism of NO3 and NO2 formation in water is the result of dissolution of nitrogen oxides formed in air plasma by gas-phase reactions of dissociated N2 and O2. Along with formation of NO2 and NO3 in the plasma treated water and the dissolution of NOx in water. As a result, in plasma activated water the predominate species are NO3 and NO2.

Functionally, components of plasma activated water with low pH have proven synergistic anti-microbial effects against bacteria and other microorganisms in the presence of reactive oxygen and nitrogen oxide intermediates. Hence, plasma activation has been shown useful in disinfecting water and sterilization of materials and living tissues. Current technologies include applying plasma itself or blowing active agents produced in plasma directly to the surface being sterilized to inactivate micro-organisms. While plasma activated water is a proven disinfectant, it also can be used as a natural stimulant to enhance seed germination and stimulate plant growth.

For the creation of reactive nitrogen oxide and stable nitrogen oxide, in particular, NO3 and to a lesser extent NO2, in the water, both a thermal and non-thermal plasma source can be used to achieved plasma activated water for dietary consumption to treat and prevent nitric oxide-related deficiencies and symptoms.

Thermal plasma can be a unique feature in the formation dietary plasma activated water. In thermal plasma, the joule heating effect results in high gas temperature. In thermal plasma, energy is used to heat the entire gas, and temperatures often range from 10,000-100,000K (10-100 electron volts or eV).

An arc discharge have very wide operating ranges and often utilizes non-thermal plasma activation to generate NO3. In non-thermal plasma, electron temperature is usually 10,000K or 1-10 eV; however, rotational excitation temperature, ion temperature, and bulk gas temperature are all quite low and similar to room temperature. Under such conditions, high energy electrons lead to the formation of active chemical species and radicals, such as atomic oxygen (O) and hydroxyl (OH), and electronically excited oxygen. It is reported that plasma-generated radicals and ions that behave like catalysts and participate in chain reactions that promote or accelerate reaction pathways.

To generate dietary plasma activated water for nitric oxide deficiencies, a low thermal approach can be used by bubble plasma gas through a container of water. An air compressor feeding air through a reactor, which exhausts to a piping that feeds to the bottom of a container of water.

To date, all use of plasma activated water regardless of whether it is created by thermal or non-thermal plasma activation water is limited to non-human use and has not been considered for consumption for treating nitric oxide deficiencies. Cold plasma activation, in the absence of water, applied directly is reportedly found to have medical application but not through a water carrier and especially not considered for consumption for restoring nitric oxide deficiencies or treating cardiometabolic condition or treated cancer. Namely, plasma activated water is currently only used for applications such as the stimulation of plant growth, for disinfectant purposes, and for sterilization purposes, i.e, topically. Though there exist known methodologies for making purified water and generating reactive nitrogen oxide-rich water to stimulate plant growth, until now, no beneficial effects of plasma activated water have been explored, recognized, published, disclosed, or evaluated for human health, in particular, for the treatment of cardiometabolic syndrome and/or cancer through the ingestion of plasma activated water or the prevention and/or treatment of nitric oxide deficiencies.

The invention as described below is the first consideration of using plasma activated water for oral consumption to treat metabolic syndrome conditions, cancer, nitric oxide deficiencies and symptoms despite widespread awareness of both plasma activation and nitric oxide. To date, all oral drugs and oral supplements administrated for boosting nitric oxide are derived from the addition of inorganic nitrate and nitrite salts, i.e, vegetable-derived nitrate salt, vegetable juices, potassium nitrate, sodium nitrate, nitric oxide donors in a salt form, added to liquid, tablet, pill, capsules.

Plasma activated water may be produced by non-thermal or thermal gliding arc treated water in a closed bubbler system or in an open system in the which the arc crosses a continuous stream of water or the plasma activated gases is bubbled into an open water container. Here, NO is transiently produced which subsequently oxidizes to NO2 and NO3 with the formation of H2O2, ozone, and low pH water. NO2 is relatively stable in water, whereas NO3 is stable in water. In non-thermal produced water, the pH range of about 6.8-7.6 with negligible to non-detectable levels H2O2. Post-treatment resulted in NO2 levels between 25-80 ppm which can decay with time when exposed to air and NO3 levels can exceed 500 ppm and remain relatively stable, whereas H202 decays in the presence of air and H2O2's decay is accelerated with a rise in pH or temperature.

### Cardiovascular Health and Related Conditions

Cardiovascular health is a leading concern around the world. Diseases and disorders ranging from rheumatic heart disease, hypertensive heart disease, ischemic heart disease, cerebrovascular disease and inflammatory heart disease constitute a primary cause of death in both developed and underdeveloped nations. As an advocate of putting cardiovascular disease and its risk factors on the global health agenda, the World Heart Federation strongly supports initiatives addressing healthy diets and reducing obesity.

Hypertensive disease generally refers to high blood pressure of unknown origin (primary hypertension) or caused by (secondary hypertension) certain specific diseases or infections, such as tumor in the adrenal glands, damage to or disease of the kidneys or their blood vessels. High blood pressure may overburden the heart and blood vessels and cause disease.

Blood pressure is the force of blood against artery walls as it circulates through the human body. Blood pressure normally rises and falls throughout the day, but it can cause health problems if it stays high for a long time. High blood pressure is sometimes called the “silent killer” because it usually has no warning signs or symptoms: many people do not know that they have high blood pressure, hence the recommendation to have blood pressure checked regularly. According to the CDC, about 70 million American adults (29%) have high blood pressure: that is 1 in every 3 American adults. (Nwankwo T, et al. Hypertension among adults in the US: National Health and Nutrition Examination Survey, 2011-2012. NCHS Data Brief, No. 133. Hyattsville, Md.: National Center for Health Statistics, Centers for Disease Control and Prevention, US Dept of Health and Human Services, 2013). Anyone, including children, can develop high blood pressure. It greatly increases the risk for heart disease and stroke, the first and third leading causes of death in the United States. Blood pressure is an underlying cause of heart attacks, strokes and kidney disease, and the prevalence of high blood pressure is so high, it is considered the single-largest contributor to death worldwide. It is also becoming more resistant to the pharmaceutical drugs used to lower it. In fact, blood pressure remains elevated in nearly one-third of all treated hypertensive patients on pharmaceutical drugs.

Elevated blood pressure is often associated with acute and chronic health conditions and the alleviation of elevated blood pressure leads to the improvement of oxygenation of muscles, increased energy, reduced inflammation, improved infection fighting ability, reduced chronic pain, improved memory recall, enhanced metabolism of glucose, improved male erection and female excitement.

Instead of relying on prescriptions, more patients are turning to a healthier eating approach, specifically, keeping sodium intake low and making consumption of nitrate-rich vegetables and leafy greens high. This cardio-protective daily diet, known as the DASH (Dietary Approach to Stop Hypertension) Eating Plan and variations thereof such as the Mediterranean Diet, including the MIND diet, is emerging as an effective way to delay or prevent high blood pressure altogether among other vascular and age-related conditions, such as memory loss. Unfortunately, dietary compliance remains a problem among Americans especially relating to the consumption of multi-servings per day of nitrate-rich plant-based foods and diets. Oftentimes cost, time consuming meal preparation, and unwillingness to consume multi-servings of beets, leafy greens, and other nitrate-rich whole plant-based diets is the basis of poor diet or low intake of such foods.

### Nitric Oxide

The value of nitric oxide (NO) was spotlighted when the Nobel Prize was awarded in 1998 for discovery of this naturally produced cardio-protective factor by a family of enzymes in the body. Unfortunately, as people grow older and become more sedentary, the enzyme for producing NO is poorly expressed or not functional. However, plant-based diets rich in inorganic nitrate were found to replenish NO in the body. A string of clinical studies underscored that vegetables (like red beet roots) and leafy greens (such as spinach and arugula) are replete with NO3, which is reduced by oral microbiome to NO2 and further reduced to NO by low pH and hypoxic conditions in the body to NO. Diets known for promoting heart health and lowering rates of diabetes and obesity, like Japanese diets, Mediterranean diets and plant-based diets, such as DASH, among others including MIND, TLC, Ornish, and Mayo, incorporate natural whole foods that are rich in inorganic NO3.

Nitric oxide is emerging as a critically important cardioprotective and vascular wellness factor. With age, arteries lose their elasticity and ability to make nitric oxide to prevent cardiovascular disease. Scientific evidence suggests that vascular aging may be delayed through the increased production of nitric oxide, thereby, enhancing cardiovascular function. Athletes have already taken advantage of these discoveries by increasing their consumption of leafy greens and beetroots, both of which are rich in nitrate, to elevate nitric oxide levels that increase endurance and performance.

A study (Apr. 15, 2013) conducted by researchers from Queen Mary University of London, and published in the American Heart Association Journal, Hypertension, provides further support for the importance of nitric oxide and of the blood pressure lowering effects on nitrate-rich vegetables. (Enhanced vasodilator activity of nitrite in hypertension: critical role for erythrocytic xanthine oxidoreductase and translational potential. Ghosh S M et al. Hypertension. 2013 May; 61(5):1091-102). Subjects who drank beetroot juice containing a natural source of inorganic nitrate show an average 10-point decrease in their blood pressure. Sustaining such levels may be critical in maintaining normal blood pressure. The lead author, Dr. Ahluwalia, reported to Medical Xpress: “our hope is that increasing one's intake of vegetables with a high dietary nitrate content, such as green leafy vegetables or beet root, might be a lifestyle approach that one could easily employ to improve cardiovascular health.”

Reducing hypertension would not only improve health outcomes for individual patients but would also benefit the health system as a whole. Although the percentage of resistance to antihypertensive drugs is relatively lower in the U.S., elevated blood pressure among a rapidly growing number of baby boomers will mean more challenges for health care in the long run unless appropriate measures and lifestyle changes are designed in put in place. Poor diet and physical inactivity remain the primary drivers of cardiovascular disease and metabolic disorders such as gout, obesity, hypertension, and diabetic insulin resistance. A daily lifestyle of a cardioprotective diet, including nitrate-rich plant-based foods, is a solution. More specifically, the amino acid, arginine can be a source for the body to make nitric oxide. Unfortunately, the aging process results in the loss our ability to make nitric oxide through the use of arginine, thereby increasing reliance on a non-arginine source, specifically, dietary nitrate. Plant-based foods, such as the DASH Diet, rich in leafy greens such as arugula, kale, and spinach, help supplement the body's pool of nitrate, which can be converted to nitric oxide, independent of arginine.

But a healthful eating strategy alone will not mean better outcomes for patients without a model to help them break bad habits and support dietary changes on a personal level, one day at a time. Indeed, even if individuals and communities have a commitment to healthy diets, the necessary resources may not be readily available due to economics or convenience. For example, some communities may be described as ‘food deserts’, defined as a region vapid of fresh fruit, vegetables, and other healthful whole foods; often times such food deserts are found in impoverished areas. In other situations, individuals may need to rely on convenience food to ensure sufficient intake of nitrate-rich nutrition, and in such environments, it would be beneficial to have access to nutritional supplements that easy to ingest, transport and store.

The problem is further exacerbated as multiple-daily servings are inconvenient to prepare, natural-based or whole plant-base ingredients are not always available, and high-quality nutrient-dense produce is often expensive. Hence, compliance with plant-based diets is low among Americans.

Nitrate-rich dietary supplements represent an attractive alternative, however, many of supplements are not adequate to provide a significant amount of nutrition to achieve the desired or target physiological benefit necessary or recommended. Furthermore, consumers are reluctant to consider supplementation since many supplement ingredients are too expensive, questionable and not validated, regulated, or lack clinical support of NO-mediated outcomes.

What is needed therefore, is a nitrate-rich, non-supplement-based natural source to complement plant-based foods and diet. Preferably, such a nitrate-rich complement is conveniently available, affordable, easy to ingest, palatable and free from unnecessary additives.

### SUMMARY OF THE INVENTION

Water is compatible with day to day conveniences, easy to ingest and palatable. Disclosed herein are novel water compositions and methods of producing such water, wherein the water is nitrate rich. In an embodiment, the water compositions of the invention are affordable, preferably derived from natural sources and not spiked with exogenously manufactured nitrate, nitrite salts, potassium or sodium forms. The dietary plasma activated water disclosed herein is a unique and novel source of dietary nitrate that is both naturally produced by electricity from either a DC or AC source and effective in combating cardiometabolic syndrome and nitric oxide deficiencies, including cancerous conditions.

In an embodiment, the present disclosure relates to compositions and methods that are effective for the prevention and/or treatment of unwanted cardiometabolic syndrome outcomes and symptoms of nitric oxide deficiencies.

Disclosed herein are methods and compositions for treating, preventing and managing nitric oxide deficiencies, in particular cardiovascular health complications. Such cardiovascular complications are clustered under cardio-metabolic syndrome and include a combination of risk factors such as hypertension, endothelium dysfunction, impaired fasting blood glucose, high levels of triglycerides, abdominal obesity, and weight gain that, collectively, puts us at risk for heart disease and type 2 diabetes.

As a first-line of defense these conditions can often be blunted with dietary lifestyle interventions including daily consumption of nitric oxide-potent diets such as, but not limited to, the DASH, MIND, and Mediterranean Diets among other plant-based diets rich in inorganic nitrate. Dietary nitrate supplied through plant-based, leafy greens contained within these diets are bio-converted within the body to reactive nitrogen oxide intermediates, including nitrate and nitrite, collectively, termed, NOx, to form a natural cardio-protective and vasodilator (or blood vessel relaxation factor), nitric oxide, NO. Unfortunately, ensuring elevated, sustained NO in the body is hampered by poor daily compliance to plant-based diets, consisting primarily of multiple servings of leafy greens and other nitric oxide-potent vegetables and fruit. Likewise, dietary nitric oxide supplements often fail to achieve a NO-beneficial effect because consumers are reluctant to consume such supplements due to questionable claims and poorly validated ingredients that lack clinical support. To overcome these limitations and problems, the specific embodiments of the present invention comprise (1) methods of generating nitrate-rich water without the addition of exogenously added salts, such as potassium nitrate or sodium nitrate, through plasma activation of pure water by applying high voltage arc at the water-air interface (i.e. in a closed system consisting of bubbler or open system consisting of a water flow crossing an electric arc) through either AC or DC-driven positive transient spark or arc discharge at the air-water droplet interface resulting in dietary plasma activated water, and (2) the use of dietary plasma activated water for the treatment, prevention and/or managing cardio-metabolic syndrome wellness outcomes, specifically, maintaining normal blood pressure, reducing elevated triglycerides among other cardio-metabolic factors that contribute to weight gain, type 2 diabetes, and cardiovascular complications as well as cancerous growths.

As disclosed herein, dietary plasma activated water can be used for several nitric oxide deficiencies and symptoms, including, but not limited to an oral rinse for the management of tooth decay, such as caries, and periodontitis to topical application for the treatment of skin fungus and acne and precancerous conditions. Furthermore, consumption of plasma activated water can alleviate fatigue, low energy, decreased workout potential, low stamina and endurance, erectile dysfunction or decreased libido, poor memory or concentration, irritability, depression, or anxiety, insomnia or difficulty sleeping, asthma or poor lung function.

### BRIEF DESCRIPTION OF THE DRAWINGS

The drawing represents the method of orally consuming plasma activated purified water to increase blood flow to the brain and heart, lower elevated blood pressure, improve peripheral endothelial function, increase glucose metabolism, promote local and systemic anti-microbial activity, lessen cardiometabolic syndrome conditions and treat symptoms of nitric oxide deficiencies.

### DETAILED DESCRIPTION OF THE INVENTION

The following detailed description is exemplary and explanatory and is intended to provide further explanation of the present disclosure described herein. Other advantages, and novel features will be readily apparent to those skilled in the art from the following detailed description of the present disclosure.

In an embodiment, provided herein are novel methods for improving health, including alleviating cardiovascular complications comprising the consumption of plasma activated water by a subject in need thereof. In an embodiment, consumption of plasma activation of purified water results in bio-convertible nitrate-rich water that is achieved independently of exogenously added sources of nitrate salts. In an embodiment, plasma activated water suitable for dietary purposes is made by the process of subjecting water to an electric charge. For example, following the application of a high voltage arc across a water spray or within a bubbler, a NOx-rich water may be produced with the bio-equivalency of >5-7 mM of NO3 similar to that of a plant-based, nitrate-rich leaf green diet, such as the NIH DASH diet. Such water may be useful for the prevention of hypertension.

In certain embodiments, methods of producing plasma activated water comprise the use of a high voltage plasma activated water device that includes an open reaction chamber to allow ambient air near the water flow. In an embodiment, the device may optionally include: an air inlet into the open reaction chamber to increase N2 near the reaction space and air-water interface water flow, a pump powered by DC or AC to supply the water flow, a ground electrode and reaction electrodes powered by DC or AC, where the water inlet and the water outlet configured as a spray and/or continuous water drop flow (or stream) in the reaction chamber. In certain embodiments, the reaction electrodes may be in juxtaposition near the opening to ensure plasma activation at the air-water droplet, or configured where the electrode is positioned at the air-water interface and the ground electrode is submerged in the water undergoing vortex (ie, static mixer, a venture mixer, or a cyclone mixer for bubbling) generating aeration of the water, and a plasma activated water reservoir collection device. The collection device can be any suitable container such as but not limited to a drinking bottle, water storage bottle, gallon etc, that receives the plasma activated water from the reaction.

In an embodiment, a high voltage plasma activated water device that can achieve output voltage between 4K to 400K voltages as an electrical arch is provided that includes an open reaction chamber to allow air near the reaction space and air-water interface water flow. In such an embodiment, water may be provided in a variety of ways: an outlet may be configured to distribute water as a spray, continuous water drop, flow or stream. In certain embodiments, the water supply may involve the delivery of water droplets in the reaction chamber comprising the use of one or more pipette-like structures/apparatuses to generate water drop(s), wherein the water drops comprise a maximal surface area compared to the mean diameter (of the droplet) typically approximately 10-15 micron (1 micron= 1/1000 mm) varying in size from 1 to 100 micron diameter. The device may be configured such that it can be adjusted with higher input voltage, where the reaction electrodes are optionally juxtaposed perpendicular and across from one another by a distance of 5-20 mm, and a distance from the water outlet of 2-10 mm to ensure the water flows as a drop or as a continuous droplet stream and such that the water crosses the arc thereby ensuring plasma activation at the air-water droplet. The device may be configured with a collection device such as a bottle, gallon or container for receiving the plasma activated water.

In certain aspects of the invention the electrodes produce reactive nitrogen in the plasma activated water with higher voltage arch applied across the air-water interface.

In certain other aspects of the invention, the plasma activated water made as described herein may be utilized to promote plant growth and seed germinating activity.

In another aspect of the invention, the plasma activated water of the invention may be utilized to inhibit the growth of infectious organisms, including but not limited to, streptococci and lactobacilli, the causative agents of caries and tooth decay. As known to those skilled in the art, high counts of mutans streptococci and/or lactobacilli indicate a high caries risk status. If protective factors cannot take effect, carious lesions will develop. As demonstrated herein, dietary plasma activated water is effective in reducing and/or eliminating cariogenic bacteria.

In another aspect of the invention, the plasma activated water of the invention may be utilized to inhibit the growth of tumor and pre-cancerous growth in subjects. The use of plasma activated water in this capacity may be beneficial for a variety of cancers including but not limited to, colon, bladder, stomach, as well as vascularized tumors found for example, in the lung, brain, bone, liver, pancreas, among other organs and glands. Though not wishing to be bound by the following theory, it is known that nitrate and nitrite ingestion contribute to cancer through the formation of nitrosamines. For decades, nitrosamine has been deemed carcinogenic by regulatory agencies based on preclinical studies. However, acidified nitrate has been reported on to inhibit bacterial and transformed cell growth in vitro; such nitrate and/or nitrite salts have not been considered in vivo and certainly not the nitrate generated in plasma activated water. Surprisingly, the present invention addresses this issue by showing that nitrate and nitrite containing plasma activated water, when ingested can be used for the alleviation cancer and prevention of tumor formation. This invention claims the use of nitrate and nitrite containing plasma activated water is anti-tumorgenic and can prevent the growth of transformed cells. The findings demonstrated herein show that the novel use of plasma activated water is uniquely non-obvious, and unexpected given that nitrate and nitrite are labeled carcinogens and would not be consider as a cancer treatment modality based on the clinical evidence thus far, but instead would be promoters of cancer.

In a particular embodiment, plasma activated water was generated using a commercially available high-voltage generator, and a modified Tesla coil. A small high efficiency module released a high-voltage pulse output with simple external circuit power supply consisting of an input voltage of DC 3.7V-7.4V (Input Current: 4 A) with an output Voltage between 400 KV-1000 KV. The arc distance of the high voltage discharge ranged between 10 mm-20 mm. The power supply was provided by either 2-4× 3.7V Lithium Batteries. The electrodes of the generator were positioned within 5 to no more than 20 mm from each other and distance was varied based on applied voltage. The resultant arc was placed 2-10 mm from the water flow whereby the water flow was directed through the midpoint of the arc. The resulting water comprised plasma activated water consisting of elevated levels of nitrates in excessive of 500 ppm, and lacking any unnecessary nitrogen-based salts, i.e, nitrate, nitrite, and related nitrogen oxide intermediate additives.

In another embodiment, plasma activated water was generated using a commercially available device, referred to as a PLASMAPONIC™ machine developed by Advanced Plasma Solutions (Pennsylvania, USA). The PLASMAPONIC™ machine is a closed system with a power requirement of 115-220 V. The machine is based upon an electric arc resulting in NO3 of 100 mg/L gallon per 4 hrs. It was developed to create water comprising oxygen and nitrogen species to be delivered exclusively to plants and/or seeds to increase root growth, reduce plant growth, and minimize water usage.

This is the first time that the PLASMAPONIC™ device has been considered as a source for plasma activated water for human health, specifically modified for consideration to create NO3 levels physiologically relevant for the prevention and/or treatment of cardiometabolic syndrome and/or restore natural nitric oxide levels, independent of plant-base diets and/or nitric oxide donors and/or nitrate and/or nitrate salts supplementation.

The PLASMAPONIC™ process loosely replicates lightning: atmospheric gas is exposed to powerful electrical or electrostatic discharges, which ionizes the individual components of atmospheric gas, into what is termed as plasma radicals. Because the primary constituents of the atmosphere are nitrogen and oxygen, most of these radical species are made up of these components. PLASMAPONIC™ in particular, utilizes non-thermal plasma. In non-thermal plasma, electron temperature is highest (usually 10,000K or 1 eV); however, rotational excitation temperature, ion temperature, and heavy particle temperature (or the bulk gas temperature) are all quite low (room temperature). Under such conditions, high energy electrons lead to the formation of active chemical species and radicals, such as atomic oxygen (O) and hydroxyl (OH), and electronically excited oxygen. It is these plasma-generated radicals and ions that behave like catalysts, and participate in chain reactions that promote or accelerate reaction pathways. On the other hand, thermal plasma is often characterized by temperature equilibrium, where the temperature of all energy levels and components are nearly equal. In thermal plasma, the joule heating effect results in high gas temperature. In thermal plasmas, energy is used to heat the entire gas, and temperatures often range from 10,000-100,000K (10-100 electron volts (eV)).

In an embodiment, the PLASMAPONIC™ system bubbles plasma gas through a container of water. There is an air compressor feeding air through a reactor, which exhausts to a piping that feeds to the bottom of a container of water. The reactor is considered to be on the lower end of the temperature spectrum for gliding arc. Here, the PLASMAPONIC™ machine was run for a longer period of time to recycle plasma activated to enrich the NO3 for human health benefit. The table below shows approximate value of the NO3 generate in a gallon of water after recycling the water to set conditions within the PLASMAPONIC™ machine. At the 6 hr time point, H2O2 was negligible and pH was 6.4-6.8.

| Time of treatment (hrs) | NO2 (ppm) | NO3 (ppm) |
|---|---|---|
| 1 | 10 | 50 |
| 2 | 15 | 150 |
| 4 | 40 | 250 |
| 6 | 60 | >350 |

NOx concentration may also vary somewhat depending on slight deviations from input voltage and electrode distance and purity of water within controlled tolerance ranges as customary within the field of art. This embodiment shows that a human health beneficial level, such as lowering blood pressure and increase time to exhaustion, was achieved after consuming plasma activated water containing 5-7 mM of NO3.

The plasma activated water of the invention comprises a novel dietary NO3 source, wherein said water is useful in achieving, maintaining and improving desired NO levels in subjects, and wherein said water may also be useful in restoring functional NO levels in nitric oxide-deficient subjects by increasing salivary bioconversion of NO3 to >200 uM NO2 with the use salivary NOx test strips 2 hours post ingestion of a plasma activated water consisting of >5 mM NO3. Plasma activated water may also lower blood pressure, and/or restore NO mediated cardiovascular benefits, including but not limited to aspects such as restoring endothelium function (i.e, flow mediated dilation). In an embodiment, the NO3 is present in a concentration in excess of 5 mM NO3 and derived solely from the plasma activation of purified water, in the absence of any other additives such as plant or salt form additions. The plasma activated water as described herein is effective in elevating the saliva conversion of NO3 to at least >200 uM NO2. Furthermore, plasma activated water as described herein is effective in improving cardiovascular health, such as by reducing systolic blood pressure. In an embodiment, the systolic blood pressure of a pre-hypertensive or hypertensive subject was reduced by approximately 2-11 mmHg within approximately 2-3 hours of drinking the plasma activated water 2 cup or approx. 16 oz (240 ml) and may be sustained within 2-11 mmHg range for a finite period of time of 3-6 hrs.

Once the water is activated, the NO3 is shelf stable. Based on the nature of the activation, i.e, thermal vs non-thermal and duration of expose, NO2 is also generated, however, with time, NO2 decomposes into NO3. And over time, if any H2O2 is generated, it decomposes to less than detectable levels. At a minimum, the critical product, NO3, is water stable at room and refrigerated temperatures.

In an embodiment, the novel compositions (also referred to herein as dietary plasma activated water) as disclosed herein, comprise plasma-generated dietary NO3, wherein said compositions are effective in modifying functional nitric oxide levels in a subject by increasing salivary bioconversion of NO3 to NO2. Such compositions may be used to establish, restore, improve and/or maintain desired nitric oxide levels.

In an embodiment methods are provided for modifying and improving NO levels in a subject, comprising administering to said subject, a composition comprising dietary NO3 in the form of plasma activated water, wherein said plasma activated water modifies functional nitric oxide levels in nitric oxide-deficient subjects by increasing salivary bioconversion of nitrate to nitrite, lowers blood pressure, and/or restores nitric oxide mediated cardiovascular benefits, including but not limited, restoring endothelium function, i.e, flow mediated dilation. Use of the compositions described herein result in improved cardiac health.

Effective dosages and schedules for intake of plasma activated water may be determined empirically, and making such determinations is within the skill in the art. The dosage ranges for the administration of the supplements are those large enough to produce the desired effect in which the nitric oxide levels are modified to acceptable or desirable levels. The dosage should not be so large as to cause adverse side effects. Generally, the dosage will vary with the age, condition, sex and extent of the nitric oxide levels desired, frequency of administration, or whether other drugs are included in the regimen, and can be determined by one of skill in the art. The dosage can be adjusted by individual users or by a physician in the event of any counter indications. Dosage can vary, and can be administered in one or more dose administrations daily, for one or several days. Guidance can be found in the literature for appropriate dosages for given classes of nutritional supplement products. To achieve a physiological beneficial effect, 5-7 mM NO3 is an adequate dose per day. However, it is not unusual to consume 3-times this amount per day with associated heart health benefits when following the NIH-supported DASH diet.

Clinical studies have shown that 5-7 mM (310-434 mg/L/day) NO3 is sufficient to reduce systolic blood pressure in hypertensives by 4-11 mmHg. In the case of the DASH diet, with 4-6 serving of nitrate-rich leafy greens the NO3 may exceed 1000 mg/L/day. Hence, for plasma activated water, a subject could achieve these values per day if so desired. By adjusting the voltage and/or expose to repeat cycling of water to an established arc gradient and/or simply diluting a concentrated plasma activated water source, the subject could adjust their per se water dose/serving to achieve a 5-7 mM dose per day with 1-6 water glasses of plasma activated water per day. The unique benefit of this method, unlike nitric oxide supplements, is that the consumer or user can both measure the NO3 prior to consumption with a simple Griess reagent test strip and make adjustments to the amount of NO3 based on consumer's daily consumption of water by diluting the water source. Plasma activated water is unique from other nitrate sources in that the consumer can make adjustments at the point of ingestion with known amounts; this cannot be accomplished with plant-base diets and prescriptive doses in supplements. In an embodiment of this invention, the level of plasma activation is determined by a semiquantitative colorimetric NO3 test strip which may be directly applied to the water. In the case for a tabletop plasma activated water machine, multiple settings can be implemented to achieved varying amounts of NO3, the initial or default setting may be set to activate a half a gallon (or 2 L) a water a day to achieve 5-7 mM which translates to 8-8 oz glasses per day. Tabletop plasma activated machine may also be supplied with NO3, NO2, pH detection strips to confirm readings of 300-500 ppm NO3, 10-40 ppm NO2, pH 6.8+/−1.5, respectively.

Since air has natural humidity, water is not added to the chamber with a gliding arc discharge and instead the air from the chamber is pumped into a contained water source, such as a bottle, bucket, or any container for water, resulting in NO3 and NO2 enriched water from the oxidation of nitric oxide gas. The plasma activated gas from the naturally humidified chamber is bubbled into the water through a tube or pipe leading, which contains an air stone to increase the surface area of water to the gas, to the contained water source. The basis for a stand-alone table top plasma activated water appliance or device will be based on this process.

Following intake of plasma activated water disclosed herein, the efficacy of the intake may be assessed in various ways well known to the skilled practitioner. For example, one of ordinary skill in the art (i.e. an athlete) will understand that plasma activated water as described herein is efficacious in treating or inhibiting symptoms associated with poor cardiovascular health and nitric oxide deficiencies. Furthermore, nitric oxide levels can be measured by methods that are known in the art, for example, using a saliva test strip to detect the bioconversion of NO3 to NO2 in the oral cavity, a necessary and critical step.

Variations contemplated in administering the subject composition to humans or other animals include, but are not limited to, providing as a single dose or as other multiple part dosages. Additionally, alternative avenues of administration besides oral administration are contemplated herein.

The following examples are given to illustrate exemplary embodiments of the present disclosure. It should be understood, however, that the present disclosure is not to be limited to the specific conditions or details described in these examples. Examples are provided below to facilitate a more complete understanding of the invention. The following examples illustrate the exemplary modes of making and practicing the invention.

### EXAMPLES

### Example 1

### Plasma Generation of Dietary Nitrates by Electrical Arc Across a Flow of Purified Water

As shown below in Table 1, the water resulting from being plasma activated, was slightly acidified water (pH<6) with 3 mg/L of H2O2 which dissipated over the course of several hours. NO3 and NO2 were immediately detected and varied based on the voltage input:

TABLE 1

| Conditions and NOx | NO3 | NO2 (mg/L) |
|---|---|---|
| Purified water | 0 | 0 |
| Purified water across an arc | 250-1,000 | 2-10 |

This example demonstrates that NOx can be generated in purified water with a high voltage coil powdered by low voltage DC source generating an electric arch across a water stream resulting in high levels of nitrate and nitrite for consumption with NO beneficial effect as shown in Example 2 and 3. The high voltage generator is a modified tesla coil, which release high-voltage pulse output with an input Voltage DC 3.7V-7.4V (Input Current: 4 A; Input Wire Length: 10 cm; Output Voltage: 800 KV-1000 KV; Output; Output Wire Length: 16 cm; Discharge Time: 10 s; Arc distance/High voltage discharge distance range: 2 mm-10 mm; Power supply: 1× 3.7V Lithium Battery, 2× 3.7V Lithium Batteries in series (such as 18650 Battery), NiCd/NiMH Batteries, 4V/6V Lead-acid Batteries, etc. Water flow between arc was controlled with a micro peristaltic water pump at a rate of 10-40 ml per min.

### Example 2

### Plasma Activated Water was Found to Inhibit S. mutans in the Oral Cavity Post Gargling

Assessment of S. mutans and Lactobacillus spp. colonization was assessed post gargling for 2 min with repeated rinses (and subsequently swallowed) purified water or purified water sprayed across plasma arc (Example 1). After 2 hrs, saliva samples were taken both group and the CRT test was used.

The CRT bacteria test from Ivoclar Vivadent enables the simultaneous determination of the S. mutans and Lactobacillus spp. counts in saliva by means of selective agars. The blue mitis-salivarius-agar with bacitracin was used to detect mutans streptococci, while the light culture medium, Rogosa agar, was used to evaluate Lactobacillus. Foils protected the agars from drying out and contamination. The deep indentation in the carriers prevented the culture media from slipping out.

The agar plates were incubated at 37° C. for 2 days in a CO2 atmosphere (CRT incubator), after added with a tablet of NaHCO₃ to stimulate bacterial growth, following which the total count was performed. The NaHCO₃ tablet placed in the test vial releases CO2 when it comes into contact with moisture. This creates favorable conditions for bacterial growth.

S. mutans occurred as small blue colonies with a diameter of <1 mm on the blue agar, while Lactobacillus spp. were detected as white colonies on the transparent agar. The comparison with the corresponding pictures in the model chart permitted the assessment of the caries risk. In this context, counts higher than 105 CFU of S. mutans and/or Lactobacillus per milliliter of saliva indicating a high/lower risk for dental caries.

The bacteria count from the purified water was too numerous to count, whereas 2-4 colonies were detected post dietary plasma activated water treatment.

This example is the first demonstration that plasma activated water exhibits anti-bacteria activity in the mouth as shown by the reduction of the caries promoting bacteria.

### Example 3

### Effects of Ingesting Plasma Activated Water on Systolic BP in Prehypertensive Subject

Post gargling in Example 2, water was swallowed and evaluated for saliva bio-conversion and blood pressure (BP). Systolic blood pressure (SBP) SBP and saliva nitric oxide status were evaluated 2 hours post ingestion of plasma activated water containing approximate 2.5-3 mmol NO3 which is less than the desired target of 5-7 mmol NO3 to elicit a blood pressure lowering effect. An 2-3 mmHg reduction was found in SBP 2 hours post-consumption of 200 ml (approximately 166 mg/L NO3) of plasma activated water. As measured with NO Berkeley Test Strips® salivary NO2 levels increased from <20 uM to nearly 220 uM during this same time period. The subject evaluated was relatively healthy, non-medicated and prehypertensive (125-130 mmHg). Prior to consumption, 150 mg vitamin C was add to the plasma activated water.

| Treatment | saliva | SBP |
|---|---|---|
| Pre-plasma activated water | 0-20 uM | baseline |
| 2 hrs post plasma activated | 100-220 uM | Reduction by 2-3 mmHg |
| water | | |

This example is the first demonstration that plasma activated water is converted by the oral microbiome to NO2 (as demonstrated Berkeley Test) and was subsequent reduced to NO resulting in the lowering of blood pressure.

Having described the invention with reference to a particular method and composition to reduce caries promoting bacteria in situ and improving cardiovascular fitness with the reduction of systolic blood pressure post ingestion of NOx-rich plasma activated water, it will be apparent to those of skill in the art that it is not intended that the invention be limited by such illustrative embodiments, and that modifications can be made without departing from the scope or spirit of the invention. It is intended that all such obvious modifications and variations be included within the scope of the present invention as defined in the appended claims. The claims are meant to cover the claimed components and steps in which is effective to meet the objectives there intended, unless the context specifically indicates to the contrary. This invention is the first report to demonstrate that a single and stand-alone device to rapidly and inexpensively produce NOx-containing water and a source for the body to make nitric oxide results in lifestyle changes that improves oral and cardiovascular health. The uniqueness of the invention is reflected in the simplicity and ease of use of the device and method that allows the bases of a device designed as a kitchen appliance, such as a soda stream carbonation device to make sparking water or a Nespresso coffee maker to make coffee. In this instance, a simple high voltage generated supplied by a AC or DC power source with a water reservoir and pump (similar to that found in a Nespresso coffee device) would provide NOx water intended to improve oral and cardiovascular health.

In like fashion, plasma activated water can be created in a water bottling plant prior to filling disposable water bottles with a defined amount of dietary inorganic nitrate independent of adding exogenous plant-extract or nitrate salts in the form of sodium or potassium, or other type of NO donors compounds.

### Example 4

### Generating Blood Pressure Lowering Bioequivalent NOx Through a Plasma Activation Table Top Appliance that Bubbles Plasma Activated Gas into an Open Water Container

Since air has natural humidity, water in this instant is not added to the plasma activation chamber which contains a gliding arc discharge. Here, plasma activated gas is generated and the air from the chamber is pumped through a tube into a water source in a bottle or any open container, resulting in NO3 and NO2 enriched water. The basis for a stand-alone table top plasma activated water appliance or device will be based on this example. The water generated under these conditions exhibit blood pressure lowering activity and endothelial restorative capabilities when consumed, ie, a single drink of an 8 oz plasma activated water from plasma activated gas results in the of ingestion of >310 mg NO3 with blood pressure lowering within 2 hrs.

| Treatment | NO (mg/L) | NO3 (mg/L) |
|---|---|---|
| Water prior to bubble | 0 | 0 |
| 1-6 hrs post bubbling | >25 | 100->500 |

1. A method of preventing or treating undesired symptoms of cardiometabolic syndrome in a subject comprising administrating to the subject in need thereof, a symptom-reducing dose amount of plasma activated water.

2. The method of claim 1, wherein the symptom is hypertension, endothelium dysfunction, impaired fasting blood glucose, high levels of triglycerides, abdominal obesity, weight gain, type 2 diabetes.

3. The method of claim 1, wherein the process of exposing aerated water to plasma energy comprises of electric discharge initiated via an AC or DC power supply in an environment containing oxygen and nitrogen.

4. The method of claim 1, wherein plasma activated water is generated from either thermal or non-thermal ionization of air in the immediate proximity to water or thermal or non-thermal ionized air is bubbled into an open water source.

5. The method of claim 1, wherein the thermal or non-thermal ionization at the air-water interface is created by a dielectric barrier discharge, a corona or pulsed corona discharge, arc, spark, or gliding arc electrical discharge; wherein the water source of the air-water interface consists of a continuous water flow, droplet, water spray crossing an electrical discharge or a closed chamber comprises a gas inlet, a water inlet, a gas and water outlet, a ground electrode and reaction electrodes where water flows between the electrodes in the form of a water flow, droplet, spray, or where one electrode is at the air-water interface and other electrode is submersed in a water vortex with aeration or bubbler.

6. The method of claim 1, wherein the thermal or non-thermal ionization by a dielectric barrier discharge, a corona or pulsed corona discharge, arc, spark, or gliding arc electrical discharge is generated in a humidified or naturally humidified chamber and the ionized or charged air is pumped through a tube, which contains an air stone, to increase the surface area of water-air, to an open container of water in which plasma activated gas is bubbled into the water source.

7. The method of claim 1, wherein the amount of plasma activated water orally administered is between 0.001 to 1 L per day.

8. The method of claim 1, wherein the plasma activated water is boiled to remove the liquid phase and the resultant water soluble solute can be administered in the form of a tablet, capsule, lozenge, suspension, suppository, gel.

9. A method for improving nitric oxide levels in subjects having symptoms of nitric oxide deficiencies comprising administrating to the human or animal subjects plasma activated water, wherein the plasma activated water is made by the process of exposing water at the water-air interface to an electrical discharge or plasma energy source or plasma activated air is generated in a naturally humidified chamber and the ionized air is pumped through a tube which contains an air stone to increased the water-air surface area to an open container of water in which plasma activated gas is bubbled into the water source results in enriching water with inorganic nitrate and nitrite.

10. The method of claim 9, wherein the oral administration of plasma activated water increases salivary bioconversion of nitrate to nitrite within 2 hours after consumption by the subject.

11. The method of claim 9, wherein the plasma activated water of nitrate and nitrite is converted into nitric oxide in the body.

12. The method of claim 9, wherein the nitric oxide deficiency is related to elevated blood pressure, endothelium dysfunction, impaired memory recall, high levels of triglycerides and cholesterol, erectile dysfunction and decrease libido, anxiety, insomnia, and irritability, type 2 diabetes, chronic inflammation, poor anti-thrombotic effect on endothelial cells and platelets, decompensated heart failure and pulmonary hypertension, infections including bacteria, viral, and parasitic, fungal, both orally and/or systemically and/or topically, including skin application, and metastatic tumor cells, precancerous and/or cancerous transformed epithelium in the GI tract, including, but not limited to, mouth, stomach, colon, vascularized tumors in lung, pancreas, liver, kidney, testicles, ovaries, brain, and bone.

13. The method of claim 9, wherein the process of exposing water to plasma energy comprises exposing the water-air interface to an electric discharge initiated via a AC or DC power supply in an environment containing oxygen and nitrogen; wherein plasma activated water created by either thermal or non-thermal electrical discharge of the air in the immediate proximity to a water source via a closed bubbler chamber or open system consisting of a continuous water; wherein the thermal or non-thermal electrical source for ionization at the air-water interface is created by a dielectric barrier discharge, a corona or pulsed corona discharge, arc, spark, or gliding arc electrical discharge; wherein the water source of the air-water interface consists of a continuous water droplet, water spray crossing an electrical arc or a closed chamber comprises a gas inlet, a water inlet, a gas and water outlet, a ground electrode and reaction electrodes where water flows between the electrodes in the form of a droplet, spray, or where one electrode is at the air-water interface and other electrode is submersed in a water vortex with aeration or bubbler; wherein the thermal or non-thermal ionization by a dielectric barrier discharge, a corona or pulsed corona discharge, arc, spark, or gliding arc electrical discharge is generated in a humidified or naturally humidified chamber and the ionized or charged air is pumped through a tube containing an air stone to increase the surface area of air-water to an open container of water in which plasma activated air or ionized air is bubbled into the water source resulting in physiological nitrate and nitrite levels for treatment dosing of nitric oxide deficiencies.

14. The method of claim 9, wherein nitrate is present in a concentration of 100-1,500 mg/L in the plasma activated water.

15. The method of claim 9, wherein the plasma activated water contains inorganic nitrate suitable for storage and ingestion generated exclusively from the plasma activation process.

16. The method of claim 9, wherein the plasma activated water can be diluted to 5-7 mM nitrate prior to oral administration that is subsequently converted in the mouth within 2 hrs to >200 uM nitrite.

17. The method of claim 9, wherein the amount of plasma activated water orally administered is between 0.001 to 1 L per day to restore nitric oxide deficiency.

18. The method of claim 9, wherein the plasma activated water for oral administration comprises the addition of vitamin C or ascorbic acid of a range of 70-1,000 mg per serving.

19. The method of claim 9, wherein the systolic blood pressure of a pre-hypertensive subject can be reduced by 2-11 mmHg within 3 hrs.

20. The method of claim 15, wherein the plasma activated water is diluted to achieve a nitric oxide restoring dose or boiled to remove the liquid phase and the resultant water soluble solute can be administered in the form of a tablet, capsule, lozenge, suspension, gel to a nitric oxide restoring amount.
